(19) United States
(12) Patent Application Publication
(10) Pub. No.: US 2026/0000904 A1
(43) Pub. Date: Jan. 1, 2026

(54) Applying Tumor Treating Fields (TTFields) to a Subject's Body Using Electrodes Having a Fluoropolymer Layer Disposed on Successive Strata of Nickel, Palladium, and Gold

(71) Applicant: Novocure GmbH, Baar (CH)

(72) Inventors: Stas OBUCHOVSKY, Haifa (IL); Idan NASSI, Haifa (IL); Alex RACHMAN, Haifa (IL); David SHAPIRO, Haifa (IL)

(73) Assignee: Novocure GmbH, Baar (CH)

(21) Appl. No.: 19/249,590

(22) Filed: Jun. 25, 2025

Related U.S. Application Data
(60) Provisional application No. 63/664,916, filed on Jun. 27, 2024.

(51) Int. Cl. A61N 1/40; A61N 1/04

(52) U.S. Cl. CPC A61N 1/40; A61N 1/0492

(57) ABSTRACT

Alternating electric fields (e.g., TTFields) can be applied to a subject's body using a pair of electrode assemblies, each of which includes a flexible insulating substrate and a plurality of conductive pads disposed on a front side of the flexible insulating substrate. Each of the conductive pads includes a layer of nickel, a layer of palladium disposed on a front side of the layer of nickel, and a layer of gold disposed on a front side of the layer of palladium. A plurality of flexible polymer regions made from Poly(VDF-TrFE-CtFE), Poly(VDF-TrFE-CFE), and/or Poly(VDF-TrFE-CFE-CTFE) are disposed on the front side of the conductive pads. When an AC voltage is applied between the pair of electrode assemblies, an AC current will be capacitively coupled into the subject's body, which will induce the alternating electric fields in the subject's body.

### CROSS REFERENCE TO RELATED APPLICATIONS

This Application claims the benefit of U.S. Provisional Application 63/664,916, filed Jun. 27, 2024, which is incorporated herein by reference in its entirety.

### BACKGROUND

Tumor Treating Fields (TTFields) therapy is a proven approach for treating tumors using alternating electric fields at frequencies of 50 kHz-1 MHz, more commonly 100-300 KHz. The alternating electric fields are induced by electrode assemblies (e.g., arrays of capacitively coupled electrodes, also called transducer arrays) placed on the subject's skin on opposite sides of the subject's body. More specifically, when an AC voltage is applied between opposing electrode assemblies, an AC current is capacitively coupled through the electrode assemblies and into the subject's body, which induces an alternating electric field in the target region. Notably, TTFields are only effective at treating tumors when the intensity of the alternating electric field is on the order of 1 V/cm or higher.

U.S. Pat. No. 8,715,203 depicts a prior art design for the electrode assemblies that uses a plurality of insulating ceramic discs. One side of each ceramic disc is positioned against the patient's skin, and the other side of each disc has a conductive backing. An AC voltage is applied to the conductive backings, and the AC current is capacitively coupled into the patient's body through the ceramic discs.

### SUMMARY OF THE INVENTION

One aspect of the invention is directed to a first apparatus for applying an alternating electric field to a living subject at a frequency between 50 kHz and 1 MHz. The first apparatus comprises a flexible insulating substrate, a plurality of conductive pads, at least one main conductive trace, and a plurality of flexible polymer regions. The plurality of conductive pads are disposed on a front side of the flexible insulating substrate, the front side facing the subject's body, and each of the conductive pads includes a layer of nickel, a layer of palladium disposed on a front side of the layer of nickel, and a layer of gold disposed on a front side of the layer of palladium. The at least one main conductive trace is disposed on the flexible insulating substrate in electrical contact with the plurality of conductive pads, and the at least one main conductive trace is arranged so that each of the conductive pads can be driven by an electrical signal. Each of the plurality of flexible polymer regions is disposed on a front side of a respective one of the conductive pads, and each of the flexible polymer regions comprises at least one of Poly(VDF-TrFE-CtFE), Poly(VDF-TrFE-CFE), and Poly(VDF-TrFE-CFE-CTFE).

In some embodiments of the first apparatus, the layer of nickel is electroless nickel, the layer of palladium is electroless palladium, and the layer of gold is immersion gold. In some embodiments of the first apparatus, each of the conductive pads further includes a layer of copper, and the layer of copper is disposed between the layer of nickel and the front side of the flexible insulating substrate. In some embodiments of the first apparatus, each of the flexible polymer regions is printed, sprayed, or cast directly onto the plurality of conductive pads.

Some embodiments of the first apparatus further comprise a plurality of thermistors and a plurality of secondary conductive traces. The plurality of thermistors are positioned on a rear side of the flexible insulating substrate, and each of the plurality of thermistors is in thermal contact with a respective one of the plurality of conductive pads. The plurality of secondary conductive traces are configured to interface with the plurality of thermistors. Optionally, in these embodiments, a reflow process is used to attach each of the thermistors to a respective one of the secondary conductive traces.

In some embodiments of the first apparatus, the flexible insulating substrate comprises a polymer. In some embodiments of the first apparatus, the flexible insulating substrate comprises polyimide.

In some embodiments of the first apparatus, each of the flexible polymer regions has a thickness of less than 20 μm in a front-to-rear direction. In some embodiments of the first apparatus, each of the flexible polymer regions has a thickness of less than 10 μm in a front-to-rear direction. In some embodiments of the first apparatus, each of the flexible polymer regions has a thickness of less than 5 μm in a front-to-rear direction. In some embodiments of the first apparatus, each of the flexible polymer regions has a dielectric constant that is greater than 10 at at least one frequency between 50 kHz and 1 MHz. In some embodiments of the first apparatus, each of the flexible polymer regions has a dielectric constant that is greater than 20, or greater than 40, at at least one frequency between 50 kHz and 1 MHz. In some embodiments of the first apparatus, each of the layers of gold has a thickness of at least 50 nm in a front-to-rear direction. In some embodiments of the first apparatus, each of the layers of gold has a thickness of at least 75 nm in a front-to-rear direction.

In some embodiments of the first apparatus, each of the layers of gold has a thickness of 50-150 nm (e.g., 75-150 nm) in a front-to-rear direction, and each of the layers of palladium has a thickness of 100-150 nm in the front-to-rear direction. In some embodiments of the first apparatus, each of the layers of gold has a thickness of 50-100 nm in a front-to-rear direction, and each of the layers of palladium has a thickness of 100-150 nm in the front-to-rear direction. In some embodiments of the first apparatus, each of the layers of gold has a thickness of 75-100 nm in a front-to-rear direction, and each of the layers of palladium has a thickness of 100-150 nm in the front-to-rear direction.

In some embodiments of the first apparatus, each of the conductive pads has a respective area, and the areas of the plurality of conductive pads collectively add up to at least 10 cm². In some embodiments of the first apparatus, each of the conductive pads has a respective area, and the areas of the plurality of conductive pads collectively add up to at least 25 cm².

Some embodiments of the first apparatus further comprise a flexible third layer positioned behind the flexible insulating substrate. The flexible third layer has a front face, and at least a portion of the front face of the third layer is coated with an adhesive. A first region of the adhesive is positioned directly behind the flexible insulating substrate and supports the flexible insulating substrate. And a second region of the adhesive is positioned outwardly with respect to the first region and is configured to (a) when pressed against a region of skin, adhere to the skin and hold the plurality of flexible polymer regions adjacent to the skin, and (b) be easily removable from the skin.

Another aspect of the invention is directed to a second apparatus for applying an alternating electric field to a living subject at a frequency between 50 kHz and 1 MHz. The second apparatus comprises a flexible insulating substrate, a plurality of conductive pads, at least one main conductive trace, and a plurality of flexible polymer regions. The plurality of conductive pads are disposed on a front side of the flexible insulating substrate, the front side facing the subject's body, and each of the conductive pads includes, a layer of nickel, a layer of palladium disposed on a front side of the layer of nickel, and a layer of gold disposed on a front side of the layer of palladium. The at least one main conductive trace is disposed on the flexible insulating substrate in electrical contact with the plurality of conductive pads, and the at least one main conductive trace is arranged so that each of the conductive pads can be driven by an electrical signal. Each of the plurality of flexible polymer regions, is disposed as a flexible polymer layer on a front side of a respective one of the conductive pads, and each of the flexible polymer regions has a dielectric constant that is greater than 10 at at least one frequency between 50 kHz and 1 MHz.

In some embodiments of the second apparatus, the layer of nickel is electroless nickel, the layer of palladium is electroless palladium, and the layer of gold is immersion gold. In some embodiments of the second apparatus, each of the conductive pads further includes a layer of copper, and the layer of copper is disposed between the layer of nickel and the front side of the flexible insulating substrate. In some embodiments of the second apparatus, each of the flexible polymer regions is printed, sprayed, or cast directly onto the plurality of conductive pads.

Some embodiments of the second apparatus further comprise a plurality of thermistors and a plurality of secondary conductive traces. The plurality of thermistors are positioned on a rear side of the flexible insulating substrate, and each of the plurality of thermistors is in thermal contact with a respective one of the plurality of conductive pads. The plurality of secondary conductive traces are configured to interface with the plurality of thermistors. Optionally, in these embodiments, a reflow process is used to attach each of the thermistors to a respective one of the secondary conductive traces.

In some embodiments of the second apparatus, the flexible insulating substrate comprises a polymer. In some embodiments of the second apparatus, the flexible insulating substrate comprises polyimide.

In some embodiments of the second apparatus, each of the flexible polymer regions has a thickness of less than 20 μm in a front-to-rear direction. In some embodiments of the second apparatus, each of the flexible polymer regions has a thickness of less than 10 μm in a front-to-rear direction. In some embodiments of the second apparatus, each of the flexible polymer regions has a thickness of less than 5 μm in a front-to-rear direction. In some embodiments of the second apparatus, each of the flexible polymer regions has a dielectric constant that is greater than 20, or greater than 40, at at least one frequency between 50 kHz and 1 MHz. In some embodiments of the second apparatus, each of the layers of gold has a thickness of at least 50 nm in a front-to-rear direction. In some embodiments of the second apparatus, each of the layers of gold has a thickness of at least 75 nm in a front-to-rear direction.

In some embodiments of the second apparatus, each of the layers of gold has a thickness of 50-150 nm (e.g., 75-150 nm) in a front-to-rear direction, and each of the layers of palladium has a thickness of 100-150 nm in the front-to-rear direction. In some embodiments of the second apparatus, each of the layers of gold has a thickness of 50-100 nm in a front-to-rear direction, and each of the layers of palladium has a thickness of 100-150 nm in the front-to-rear direction. In some embodiments of the second apparatus, each of the layers of gold has a thickness of 75-100 nm in a front-to-rear direction, and each of the layers of palladium has a thickness of 100-150 nm in the front-to-rear direction.

In some embodiments of the second apparatus, each of the conductive pads has a respective area, and the areas of the plurality of conductive pads collectively add up to at least 10 cm². In some embodiments of the second apparatus, each of the conductive pads has a respective area, and the areas of the plurality of conductive pads collectively add up to at least 25 cm².

Some embodiments of the second apparatus further comprise a flexible third layer positioned behind the flexible insulating substrate. The flexible third layer has a front face, and at least a portion of the front face of the third layer is coated with an adhesive. A first region of the adhesive is positioned directly behind the flexible insulating substrate and supports the flexible insulating substrate. And a second region of the adhesive is positioned outwardly with respect to the first region and is configured to (a) when pressed against a region of skin, adhere to the skin and hold the plurality of flexible polymer regions adjacent to the skin, and (b) be easily removable from the skin.

### BRIEF DESCRIPTION OF THE DRAWINGS

FIG. 1 depicts a plan view and a cross-sectional side view of a first embodiment of an electrode assembly for applying TTFields to a subject's body.

FIG. 2 depicts a plan view and a cross-sectional side view of the same electrode assembly depicted in FIG. 1, but with a different cross-sectional slice through the electrode assembly.

FIG. 3 depicts a plan view and a cross-sectional side view of another embodiment of a flex-circuit based electrode assembly for applying TTFields to a subject's body.

FIG. 4 depicts one example of how the electrode assemblies depicted in FIG. 1 can be used to apply TTFields to a subject's body.

Various embodiments are described in detail below with reference to the accompanying drawings, wherein like reference numerals represent like elements.

### DESCRIPTION OF THE PREFERRED EMBODIMENTS

Although the electrode assemblies described in U.S. Pat. No. 8,715,203 are effective, those electrode assemblies are relatively stiff because they are made using solid ceramic discs with diameters on the order of 2 cm and a thickness on the order of 1 mm. And this stiffness can make it harder to position the electrode assemblies in the desired location and/or can cause a mild degree of discomfort to the patient.

One approach for overcoming this problem is to use a flexible printed circuit (a.k.a. flex circuit) to construct each electrode assembly. This could entail, for example disposing a plurality of copper printed circuit pads on a flexible insulating substrate (e.g. polyimide), with a thin insulating polymer layer disposed on top of the copper pads. In this example, each copper pad serves the same function as the conductive backing on a prior art ceramic disc, and the thin insulating polymer layer serves the same function as the insulating ceramic material of a prior art ceramic disc. More specifically, each copper pad serves as the plate of a capacitor, and the insulating polymer layer on top of that copper pad serves as the dielectric layer of the capacitor.

Because TTFields require field strengths on the order of 1 V/cm or higher to be effective and are capacitively coupled into the patient's body, it is advantageous for the capacitance of each electrode element to be as high as possible. To achieve this, because capacitance is directly proportional to the dielectric constant of the capacitor's insulating layer and inversely proportional to that layer's thickness, the dielectric constant should be as high as possible and the thickness should be as low as possible.

Most polymers have a dielectric constant of approximately 2-3. Poly(vinylidene fluoride-trifluoroethylene-chlorotrifluoroethylene), poly(vinylidene fluoride-trifluoroethylene-1-chlorofluoroethylene), and poly(vinylidene fluoride-trifluoroethylene-chlorotrifluoroethylene-chlorofluoroethylene) all have dielectric constants that are greater than 10. (These three polymers are abbreviated herein as “Poly(VDF-TrFE-CTFE),” “Poly(VDF-TrFE-CFE),” and “Poly(VDF-TrFE-CTFE-CFE),” respectively. Note that the values for dielectric constant and breakdown voltage specified herein are specified within a temperature range of 30-42° C., for example 35-42° C. or 38-41° C., and the values of those parameters outside that temperature range are less relevant.

Because Poly(VDF-TrFE-CTFE), Poly(VDF-TrFE-CFE), and Poly(VDF-TrFE-CTFE-CFE) have a high dielectric constant, one might be tempted to use these polymers as the dielectric layer in an electrode assembly for applying TTFields to a subject's body. But when any of these polymers is used to form an electrode assembly for applying TTFields to a subject's body, and the polymer is deposited directly on a conventional metal PCB pad in a TTFields electrode assembly, the underlying metal PCB pad is subject to degradation. This degradation could occur during the manufacturing process of the TTFields electrode assembly (e.g. during reflow), during use (e.g., when exposed to moisture), and/or over time after the TTFields electrode assembly has been manufactured (which would negatively impact the shelf life of the electrode assembly).

The TTFields electrode assemblies described below take advantage of the relatively high dielectric constant of Poly(VDF-TrFE-CTFE), Poly(VDF-TrFE-CFE), and Poly(VDF-TrFE-CTFE-CFE), but overcome the metal-pad-degradation problem described above.

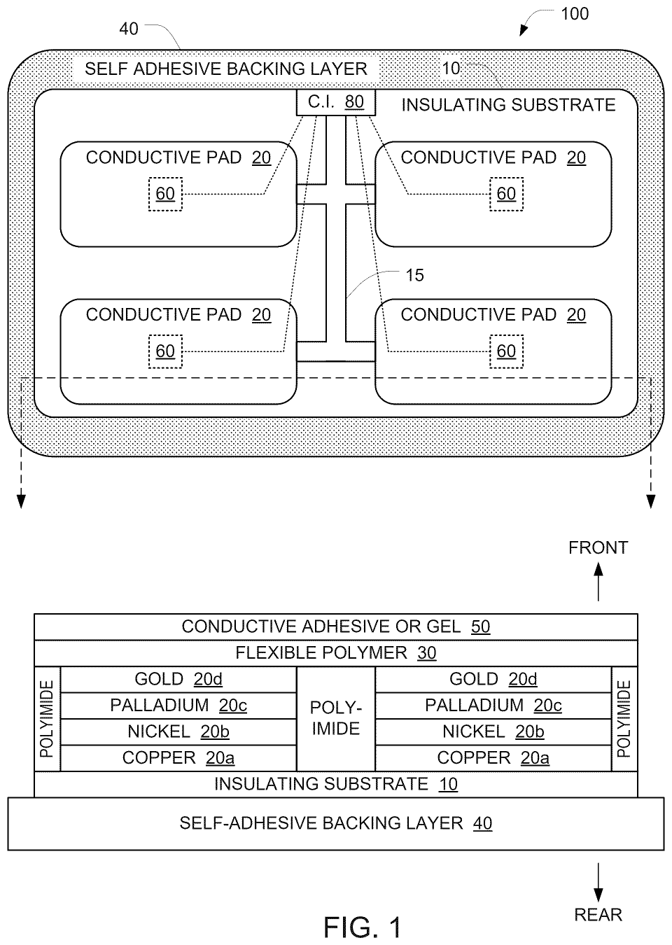

FIG. 1 depicts a plan view and a cross-sectional side view of a first embodiment 100 of a flex-circuit based electrode assembly for applying TTFields to a subject's body. In all embodiments described herein, the front of an electrode or electrode assembly is the side that faces the person's body, and the rear of the electrode or electrode assembly is the opposite side. This embodiment is used for applying an alternating electric field to a living subject at a frequency between 50 kHz and 1 MHz (e.g., 100-300 kHz).

The FIG. 1 embodiment has a flex circuit that includes a plurality of conductive pads 20 disposed on a front side of a flexible insulating substrate 10. The flexible insulating substrate 10 can be e.g., polyimide, a flexible polymer, or any other flexible insulating material (e.g., materials that are commonly used to make flex circuit substrates). Notably, each of the conductive pads 20 includes a layer of nickel 20b, a layer of palladium 20c disposed on a front side of the layer of nickel, and a layer of gold 20d disposed on a front side of the layer of palladium. In the embodiment depicted in FIG. 1, each of the conductive pads further includes a layer of copper 20a, and the layer of copper is disposed between the layer of nickel 20b and the front side of the flexible insulating substrate 10.

In some embodiments, the layer of nickel is electroless nickel, the layer of palladium is electroless palladium, and the layer of gold is immersion gold.

In some embodiments, each of the layers of gold has a thickness of at least 25 nm, at least 50 nm, at least 75 nm, at least 100 nm, at least 120 nm, at least 150 nm, or at least 200 nm in a front-to-rear direction. In some embodiments, each of the layers of gold has a thickness of 50-100 nm, 75-100 nm, 85-120, 50-150 nm, 75-150 nm, or 85-150 nm in a front-to-rear direction. In some embodiments, each of the layers of palladium has a thickness of 50-250 nm, 75-200 nm, or 100-150 nm in the front-to-rear direction.

In some embodiments, each of the layers of gold has a thickness of 50-150 nm in a front-to-rear direction, and each of the layers of palladium has a thickness of 100-150 nm in the front-to-rear direction. In some embodiments, each of the layers of gold has a thickness of 75-150 nm in a front-to-rear direction, and each of the layers of palladium has a thickness of 100-150 nm in the front-to-rear direction. In some embodiments, each of the layers of gold has a thickness of 50-100 nm in a front-to-rear direction, and each of the layers of palladium has a thickness of 100-150 nm in the front-to-rear direction. In some embodiments, each of the layers of gold has a thickness of 75-100 nm in a front-to-rear direction, and each of the layers of palladium has a thickness of 100-150 nm in the front-to-rear direction.

The thicknesses of the various layers depicted in the figures are not drawn to scale, and the layers of copper and nickel will typically be significantly thicker than the layers of gold and palladium. For example, the layer of nickel can be 1-20 μm, 1-10 μm, 2-8 μm, or 3-6 μm thick. And the layer of copper can be, e.g., 20-100 μm, 25-75 μm, 30-60 μm, or 30-40 μm thick.

Each of the conductive pads 20 has an area. At least one main conductive trace 15 is disposed on the flexible insulating substrate 10 in electrical contact with the plurality of conductive pads 20. And this at least one main conductive trace 15 is arranged so that each of the conductive pads 20 can be driven by an electrical signal. The portions of the flexible insulating substrate 10 that are not covered by either a conductive pad 20 or a main conductive trace 15 are covered with an insulating material. In the embodiment depicted in FIG. 1, this insulating material is polyimide (e.g., Kapton®, E.I. du Pont de Nemours and Company, Wilmington, DE, USA). But in other embodiments, a different insulating material can be used.

This embodiment also has a plurality of flexible polymer regions 30, each of which is disposed on a front side of a respective one of the conductive pads 20. These flexible polymer regions 30 could be regions within a single contiguous sheet of polymer material, as depicted in FIG. 1. Alternatively (not shown), these regions 30 could be discrete flexible polymer pieces that are spaced apart from each other. Each of the flexible polymer regions 30 has a front face and is disposed over and in front of a respective one of the conductive pads 20.

In some embodiments, each of the flexible polymer regions 30 comprises at least one of Poly(VDF-TrFE-CtFE), Poly(VDF-TrFE-CFE), and Poly(VDF-TrFE-CFE-CTFE). In some embodiments, each of the flexible polymer regions 30 comprises a polymer having a dielectric constant that is greater than 10 (or greater than 20, or greater than 40) at at least one frequency between 50 kHz and 1 MHz (e.g., at 200 kHz). In some embodiments, each of the flexible polymer regions 30 has a dielectric constant that is greater than 10 (or greater than 20, or greater than 40) at at least one frequency between 50 kHz and 1 MHz (e.g., at 200 kHz). The polymer region may comprise a polymer with particles having a high dielectric constant contained therein.

Because the TTFields are capacitively coupled through the electrode assembly 100, and because capacitance is inversely proportional to the thickness of the dielectric layer, the flexible polymer regions 30 are preferably thin in the front-to-rear direction (e.g., 20 μm or less, 10 μm or less, 5 μm or less, 3 μm or less, or 2 μm or less).

On the other hand, the flexible polymer regions 30 should not be too thin because that could impair manufacturability, compromise the layer's structural integrity, and risk dielectric breakdown when the AC signals are applied. In some embodiments, the flexible polymer regions 30 have a thickness that is at least 1 μm. In some embodiments the flexible polymer regions 30 are between 1-5 μm, or 1-3 μm, or 1-2 μm thick (e.g., about 2 μm), which provides a balance between the parameters noted above. The thickness of the flexible polymer regions 30 can be uniform. In alternative embodiments, the thickness can be nonuniform.

The plurality of flexible polymer regions 30 can be printed, sprayed, or cast directly onto the plurality of conductive pads 20, which makes it much easier to obtain a thin polymer layer. In some embodiments (e.g., in those embodiments where the flexible polymer regions 30 are printed, sprayed, or cast directly onto the conductive pads 20), the polymer regions have a thickness of 5 μm or less, e.g., 1-5 μm, or 1-3 μm, or 1-2 μm thick.

In some embodiments, the polymer used in the flexible polymer regions 30 can comprise VDF, TrFE, CFE and/or CTFE in any suitable molar ratio. Suitable polymers include those, for example, having 30 to 80 mol % VDF, 5 to 60 mol % TrFE, with CFE and/or CTFE constituting the balance of the mol % of the polymer. In further embodiments, the polymer comprises 40 to 70 mol % VDF, 20 to 50 mol % TrFE, with CFE and/or CTFE constituting the balance of the mol % of the polymer. In still further embodiments, VDF and TrFE constitutes 80 to 97 mol % of the polymer, and CFE and/or CTFE constitutes the remainder, i.e., 3 to 20 mol %. In other embodiments, VDF and TrFE constitutes 90 to 95 mol % of the polymer, and CFE and/or CTFE constitutes the remainder, i.e., 5 to 10 mol %. For example, the polymer can comprise 61.8 mol % VDF, 29.8 mol % TrFE, and 8.5 mol % CFE and/or CTFE.

In some embodiments, suitable polymers used in the flexible polymer regions 30 include those, for example, having 30 to 80 mol % VDF, 5 to 60 mol % TrFE, with CTFE constituting the balance of the mol % of the polymer. In some embodiments, the polymer comprises 40 to 70 mol % VDF, 20 to 50 mol % TrFE, with CTFE constituting the balance of the mol % of the polymer. In still further embodiments, VDF and TrFE constitutes 80 to 97 mol % of the polymer, and CTFE constitutes the remainder, i.e., 3 to 20 mol %. In other embodiments, VDF and TrFE constitutes 90 to 95 mol % of the polymer, and CTFE constitutes the remainder, i.e., 5 to 10 mol %. For example, the polymer can comprise 61.8 mol % VDF, 29.8 mol % TrFE, and 8.5 mol % CTFE.

In some embodiments, the polymers can have an average molecular weight of greater than 400,000 g/mol, as measured by viscometry. For example, the polymers can have an average molecular weight equal to about 413,000, as measured by viscometry at 20° C. using methyl ethyl ketone as solvent. In some embodiments, the polymers can be powder form, free of any crust or skin, before forming the polymer into the insulating layer.

Polymers comprising VDF, TrFE, CFE, and/or CTFE can be made according to methods known in the art. In some embodiments, such polymers can be prepared according to the following process. An initial mixture of VDF and TrFE (free of CFE and CTFE) can be fed into an autoclave or other suitable reactor that can be pressurized. An initiator mixed with water can be injected into the autoclave to achieve a suitable pressure, e.g., at least 80 bar, to thereby form a suspension of VDF and TrFE monomers in water. A secondary mixture comprising VDF, TrFE, and CFE and/or CTFE can then be injected into the autoclave. In some embodiments, when the polymerization reaction begins, the secondary mixture can be reinjected continuously into the autoclave such that a constant pressure of at least 80 bar is maintained.

In some embodiments, the initial mixture fed into the autoclave can comprise 25% to 95% by weight VDF (e.g., 55 to 80% by weight VDF), and 5% to 75% by weight TrFE (e.g., 20% to 45% by weight TrFE). The secondary mixture can comprise 20% to 80% VDF by weight (e.g., 35% to 70% VDF), 3% to 60% TrFE by weight (e.g., 14% to 40% TrFE), and 4% to 67% CFE and/or CTFE by weight (e.g., 7% to 34% CFE and/or CTFE). In some embodiments, the weight ratio of the initial mixture and the secondary mixture ranges from about 0.4 to about 2.

In some embodiments, the pressure inside the autoclave or reaction can be between about 80 bar and 110 bar. A reaction temperature of between 40° C. and 60° C. can be maintained. In some embodiments, the secondary mixture of VDF, TrFE, and CFE and/or CTFE can be reinjected continuously into the autoclave or reactor, for example through a gate having a non-return valve. The secondary mixture can in some embodiments be compressed using two compressors in series before being reinjected into the autoclave. As is known, the secondary mixture can be injected into the autoclave under a pressure greater than that prevailing in the autoclave, i.e., at values above 80 bar.

Other polymers comprising VDF, TrFE, CFE, and/or CTFE are also contemplated for use in the flexible polymer regions 30. For example, polymers comprising 50-80 mol % VDF, 15-40 mol % TrFE, and 2-20 mol % of CFE and/or CFTE can be used. Such polymers can have a number average molecular weight in excess of 10,000 g/mol, e.g., greater than 30,000 g/mol. Polymers of such compositions are described in U.S. Pat. No. 6,355,749, which is incorporated by reference in its entirely for its teachings of VDF, TrFE, and CFE/CTFE-containing polymers and methods of preparing them.

Increasing the total area that is covered by the conductive pads 20 will increase the capacitance of the overall device. In some embodiments, the areas of the plurality of conductive pads 20 collectively add up to at least 10 cm² (or at least 25 cm²).

The FIG. 1 embodiments can be affixed to a person's skin using a flexible third layer 40 that resembles a bandage (self-adhesive backing). In these embodiments, a flexible third layer 40 is positioned behind the insulating substrate 10. The flexible third layer 40 has a front face. At least a portion of the front face of the flexible third layer 40 is coated with an adhesive. A first region of the adhesive is positioned directly behind the insulating substrate 10 and supports the insulating substrate 10, and a second region of the adhesive is positioned outwardly with respect to the first region. (This is the portion of the flexible third layer 40 that is marked with a dotted pattern in FIG. 1 and is not covered by the insulating substrate 10.) This second region is configured to, when pressed against a region of skin, adhere to the skin and hold the plurality of flexible polymer regions 30 adjacent to the skin. The adhesive used in the second region should also be easily removable from the skin. Although the flexible third layer 40 holds the plurality of flexible polymer regions 30 adjacent to the skin, a layer of conductive adhesive or gel 50 (e.g., a tacky conductive hydrogel) may be interposed between the flexible polymer regions 30 and the skin, and the relationship between the flexible polymer regions 30 and the skin would nevertheless be considered “adjacent.” In this situation, the layer of conductive adhesive or gel 50 is disposed on the front face of each of the flexible polymer regions 30. The conductive adhesive or gel 50 is positioned to make contact with the skin when each of the flexible polymer regions 30 is being held adjacent to the skin by the second region of the adhesive. As discussed earlier herein, the flexible polymer regions 30 can be a continuous layer covering multiple (or all) conductive pads 20 or can be implemented as discontinuous regions overlying each of the conductive pads 20 separately. In either case, the conductive adhesive or gel 50 (if present) would overlie the front face of each of the flexible polymer regions 30. Herein, “conductive adhesive or gel” means “conductive adhesive or conductive gel”; and “conductive gel” may include a hydrogel.

In a variation of the FIG. 1 embodiment, a different approach is used to hold the polymer regions adjacent to the skin using a flexible third layer 40. In these embodiments, the flexible third layer 40 is configured to support the flex circuit (which includes the insulating substrate 10 and the conductive pads 20). The flexible third layer has a front face, and optionally can include a plurality of cut-out open regions that correspond to the positions of the conductive pads 20. A first portion of the front face of the flexible third layer 40 is coated with an adhesive that adheres to human skin and is easily removable from the skin. This first portion is positioned outwardly with respect to the insulating substrate 10 such that when the first portion is pressed against a region of skin, the adhesive on the first portion will adhere to the skin and hold the plurality of flexible polymer regions 30 adjacent to the skin. As in the previous embodiments, a layer of conductive adhesive or gel 50 (e.g., a layer of tacky conductive hydrogel) may be disposed on the front face of each of the flexible polymer regions 30. The conductive adhesive or gel 50 is positioned to make contact with the skin when each of the flexible polymer regions 30 is being held adjacent to the skin by the adhesive.

A plurality of thermistors 60 may be incorporated into this FIG. 1 embodiment. One way to accomplish this is to position the plurality of thermistors 60 on the rear side of the flexible insulating substrate 10, (i.e., between the insulating substrate 10 and the flexible third layer 40), with each of the plurality of thermistors 60 in thermal contact with a respective one of the plurality of conductive pads 20. In these embodiments, the flex circuit further includes a plurality of secondary conductive traces (shown in dashed lines) that are configured to interface with the plurality of thermistors 60. In some embodiments, a reflow process is used to attach each of the thermistors 60 to a respective one of the secondary conductive traces.

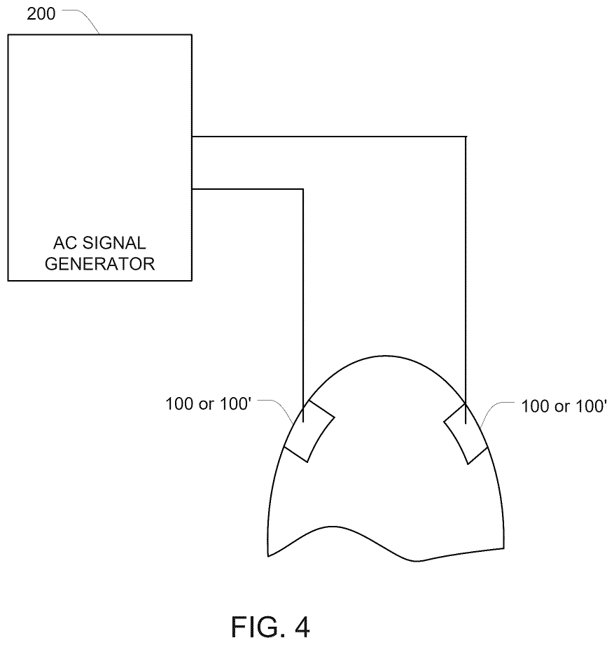

In the embodiment depicted in FIG. 1, the secondary conductive traces (shown in dashed lines) and the main conductive traces 15 terminate on a cable interface 80, and a cable (not shown) extends from that cable interface 80 to the AC signal generator (shown in FIG. 4) that applies the AC voltage to the electrode assemblies 100.

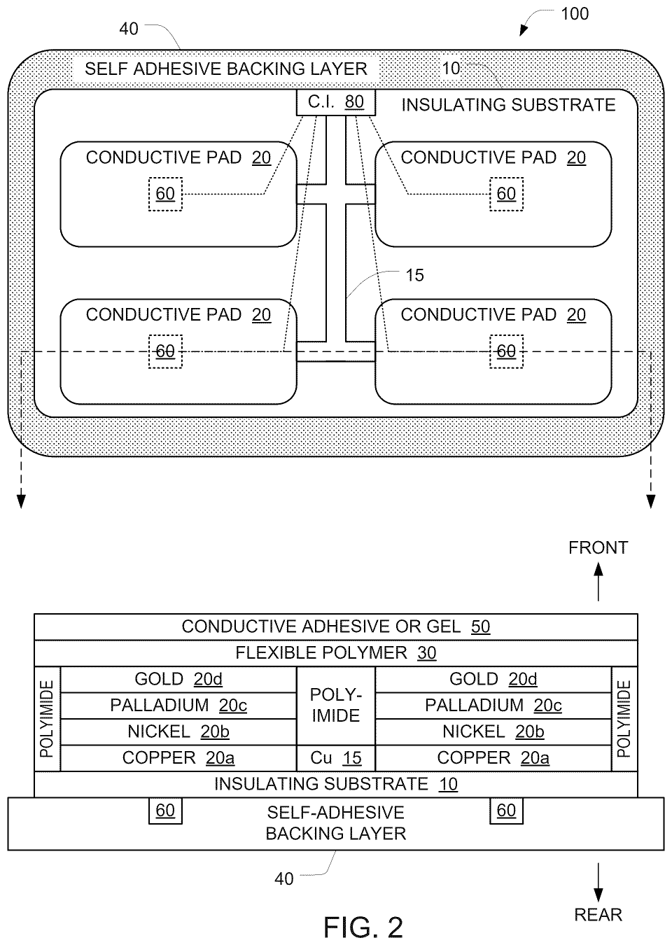

FIG. 2 depicts a plan view and a cross-sectional side view of the same electrode assembly 100 depicted in FIG. 1, but with a different cross-sectional slice through the electrode assembly. More specifically, the cross-sectional view depicted in FIG. 2 is similar to the cross-sectional view depicted in FIG. 1, except that the FIG. 2 view also passes through the conductive traces 15 and the thermistors 60. The thermistors 60 extend below the insulating substrate 10, and the self-adhesive backing layer 40 is either shaped with small wells to make room for the thermistors 60, or deforms to make room for the thermistors 60. Note that while FIG. 2 depicts the thickness of the copper of the conductive traces 15 as being the same as the thickness of the copper layer 20a, the thickness of those different regions of copper is not necessarily the same. For example, the conductive traces 15 can be thinner than the copper layer 20a that is used as the rearmost layer of the conductive pads 20.

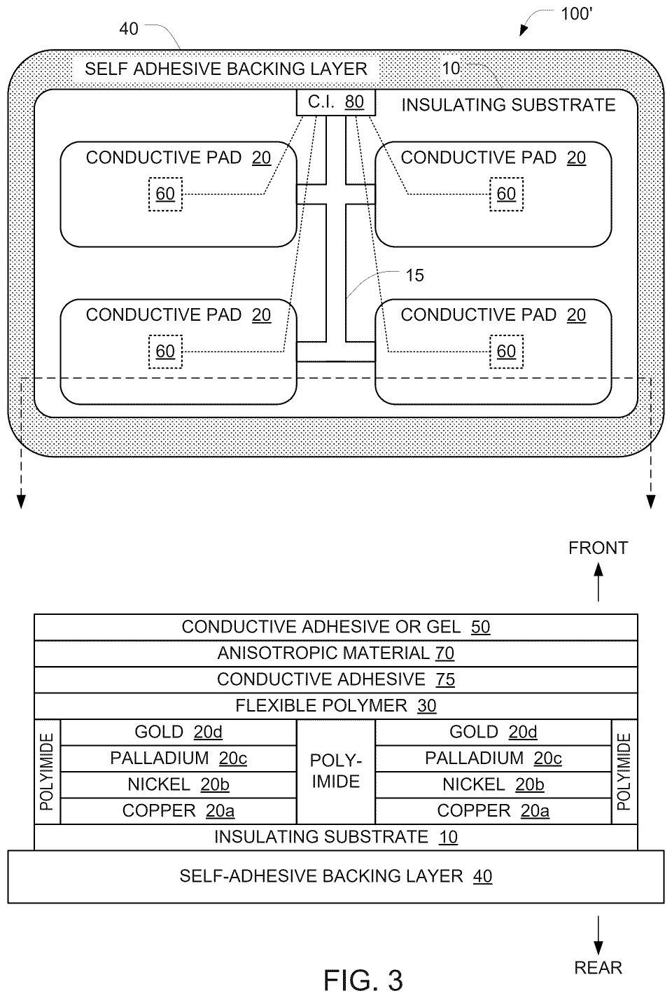

FIG. 3 depicts a plan view and a cross-sectional side view of another embodiment 100′ of a flex-circuit based electrode assembly for applying TTFields to a subject's body. This embodiment is similar to the FIG. 1 embodiment described above, except that it includes two additional layers positioned between the flexible polymer regions 30 and the conductive adhesive or gel 50. More specifically, a layer of conductive adhesive 75 is disposed on and in front of the flexible polymer regions 30, and a layer of anisotropic material 70 (e.g., a sheet of graphite) is disposed on and in front of the layer of conductive adhesive 75. The conductive adhesive or gel 50 is disposed on and in front of the layer of anisotropic material 70.

The use of anisotropic materials in electrode assemblies is described in Patent Application Pub. No. US 2023/0037806A1, which is incorporated herein by reference in its entirety. The layer of anisotropic material 70 may have anisotropic thermal properties and/or anisotropic electrical properties. If the layer of anisotropic material 70 has anisotropic thermal properties (for example, greater thermal conductivity in the plane of the layer than through the plane of the layer), then the layer spreads the heat out more evenly over a larger surface area. If the layer of anisotropic material 70 has anisotropic electrical properties (for example, greater electrical conductivity in the plane of the layer than through the plane of the layer), then the layer spreads the current out more evenly over a larger surface area. In each case, this can lower the temperature of any hot spots and raises the temperature of the cooler regions when a given AC voltage is applied to the electrode assemblies. Accordingly, the current can be increased (thereby increasing the therapeutic effect) without exceeding the safety temperature threshold at any point on the subject's skin.

In some embodiments, the layer of anisotropic material 70 is anisotropic with respect to electrical conductivity properties. In some embodiments, the layer of anisotropic material 70 is anisotropic with respect to thermal conductivity properties. In some preferred embodiments (e.g., when the layer of anisotropic material 70 comprises graphite, such as a sheet of pyrolytic graphite), the layer of anisotropic material 70 is anisotropic with respect to both electrical conductivity properties and thermal conductivity properties. In some embodiments, the layer of anisotropic material 70 is a sheet of graphite. In some embodiments, the layer of anisotropic material 70 is a sheet of pyrolytic graphite, graphitized polymer film, or graphite foil made from compressed high purity exfoliated mineral graphite.

FIG. 4 depicts one example of how the electrode assemblies 100 depicted in FIGS. 1-2 (or the electrode assemblies 100′ depicted in FIG. 3) can be used to apply alternating electric fields to a subject's body. During use, a first electrode assembly 100 is positioned on the person's skin (e.g., on one side of the tumor), and a second electrode assembly 100 is positioned on the person's skin (e.g., on the opposite side of the tumor). For example, in the context of a brain tumor positioned in the center of a person's head, the first electrode assembly 100 could be positioned on the right side of the person's head, and the second electrode assembly 100 could be positioned on the left side of the person's head.

For both of the electrode assemblies 100, the front of the electrode assembly 100 faces the person's skin, which means that the flexible polymer regions 30 face the person's skin. When pressed against the skin, the second region of the adhesive near the periphery of the flexible third layer 40 adheres to the skin and holds the flexible polymer regions 30 adjacent to the skin. When the layer of conductive adhesive or gel 50 is provided, the conductive adhesive or gel is disposed between the flexible polymer regions 30 and the person's skin. In other embodiments, in which the layer of conductive adhesive or gel is omitted, the flexible polymer regions 30 will rest directly on the person's skin.

After the electrode assemblies 100 have been adhered to the person's skin, the AC signal generator 200 applies an AC voltage between the electrode assemblies 100. Each of the conductive pads 20 acts as a capacitor's plate, and the flexible polymer regions 30 act as a capacitor's insulating layer. So when the AC voltage is applied between the first and second electrode assemblies 100, an AC current will be capacitively coupled into the subject's body, which will induce an alternating electric field (i.e., TTFields) in the subject's body.

The duration of time that the alternating electric field is applied to the target region will vary depending on the condition treated. For example, the duration of time can be determined based on when a therapeutically significant portion of the rapidly dividing cells die. Further details of the treatment method are described in U.S. Pat. Nos. 7,016,725 and 7,565,205, each of which is incorporated herein by reference in its entirety.

In alternative embodiments, a different polymer that provides a high level of capacitance may be used to form the flexible polymer regions 30 instead of the Poly(VDF-TrFE-CtFE), Poly(VDF-TrFE-CFE), and/or Poly(VDF-TrFE-CFE-CTFE) described above in connection with FIGS. 1-3. In these alternative embodiments, each of the flexible polymer regions 30 is disposed as a flexible polymer layer on a front side of a respective one of the conductive pads 20, and each of the flexible polymer regions 30 has a dielectric constant that is greater than 10 at at least one frequency between 50 kHz and 1 MHz (e.g., at 200 kHz). The thickness of each of the flexible polymer regions 30 can be less than 20 μm in a front-to-rear direction (or <10 μm or <5 μm in that direction). Example of alternative polymers that can be used in place of Poly(VDF-TrFE-CtFE), Poly(VDF-TrFE-CFE), and/or Poly(VDF-TrFE-CFE-CTFE) in these alternative embodiments include the following: (1) ceramic nanoparticles mixed into at least one of Poly(VDF-TrFE), P(VDF-HFP), PVDF, or other polymers; and (2) barium titanate and/or barium strontium titanate ceramic nanoparticles mixed into at least one of Poly(VDF-TrFE), P(VDF-HFP), PVDF. In other alternative embodiments, the flexible polymer regions 30 are formed by mixing ceramic nanoparticles into at least one other polymer (i.e., a polymer not listed above in this paragraph).

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an alternating electric field to a living subject at a frequency between 50 kHz and 1 MHz, the apparatus comprising:
a flexible insulating substrate;
a plurality of conductive pads disposed on a front side of the flexible insulating substrate, the front side facing the subject's body, wherein each of the conductive pads includes a layer of nickel, a layer of palladium disposed on a front side of the layer of nickel, and a layer of gold disposed on a front side of the layer of palladium;
at least one main conductive trace disposed on the flexible insulating substrate in electrical contact with the plurality of conductive pads, wherein the at least one main conductive trace is arranged so that each of the conductive pads can be driven by an electrical signal; and
a plurality of flexible polymer regions, each of which is disposed on a front side of a respective one of the conductive pads, wherein each of the flexible polymer regions comprises at least one of Poly(VDF-TrFE-CtFE), Poly(VDF-TrFE-CFE), and Poly(VDF-TrFE-CFE-CTFE).

2. The apparatus of claim 1, wherein the layer of nickel is electroless nickel, the layer of palladium is electroless palladium, and the layer of gold is immersion gold.

3. The apparatus of claim 1, wherein each of the conductive pads further includes a layer of copper, and the layer of copper is disposed between the layer of nickel and the front side of the flexible insulating substrate.

4. The apparatus of claim 1, wherein each of the flexible polymer regions is printed, sprayed, or cast directly onto the plurality of conductive pads.

5. The apparatus of claim 1, further comprising:
a plurality of thermistors positioned on a rear side of the flexible insulating substrate, wherein each of the plurality of thermistors is in thermal contact with a respective one of the plurality of conductive pads; and
a plurality of secondary conductive traces configured to interface with the plurality of thermistors.

6. The apparatus of claim 5, wherein a reflow process is used to attach each of the thermistors to a respective one of the secondary conductive traces.

7. The apparatus of claim 1, wherein the flexible insulating substrate comprises a polymer.

8. The apparatus of claim 1, wherein the flexible insulating substrate comprises polyimide.

9. The apparatus of claim 1, wherein each of the flexible polymer regions has a thickness of less than 20 μm in a front-to-rear direction.

10. The apparatus of claim 1, wherein each of the flexible polymer regions has a thickness of less than 5 μm in a front-to-rear direction.

11. The apparatus of claim 1, wherein each of the flexible polymer regions has a dielectric constant that is greater than 10 at at least one frequency between 50 kHz and 1 MHz.

12. The apparatus of claim 1, wherein each of the layers of gold has a thickness of at least 50 nm in a front-to-rear direction.

13. The apparatus of claim 1, wherein each of the layers of gold has a thickness of at least 75 nm in a front-to-rear direction.

14. The apparatus of claim 1, wherein each of the layers of gold has a thickness of 50-150 nm in a front-to-rear direction, and
wherein each of the layers of palladium has a thickness of 100-150 nm in the front-to-rear direction.

15. The apparatus of claim 1, wherein each of the layers of gold has a thickness of 75-150 nm in a front-to-rear direction, and
wherein each of the layers of palladium has a thickness of 100-150 nm in the front-to-rear direction.

16. The apparatus of claim 1, wherein each of the conductive pads has a respective area, and wherein the areas of the plurality of conductive pads collectively add up to at least 10 cm².

17. The apparatus of claim 1, wherein each of the conductive pads has a respective area, and wherein the areas of the plurality of conductive pads collectively add up to at least 25 cm².

18. The apparatus of claim 1, further comprising a flexible third layer positioned behind the flexible insulating substrate, the flexible third layer having a front face,
wherein at least a portion of the front face of the third layer is coated with an adhesive,
wherein a first region of the adhesive is positioned directly behind the flexible insulating substrate and supports the flexible insulating substrate, and
wherein a second region of the adhesive is positioned outwardly with respect to the first region and is configured to (a) when pressed against a region of skin, adhere to the skin and hold the plurality of flexible polymer regions adjacent to the skin, and (b) be easily removable from the skin.

19. An apparatus for applying an alternating electric field to a living subject at a frequency between 50 kHz and 1 MHz, the apparatus comprising:
a flexible insulating substrate;
a plurality of conductive pads disposed on a front side of the flexible insulating substrate, the front side facing the subject's body, wherein each of the conductive pads includes, a layer of nickel, a layer of palladium disposed on a front side of the layer of nickel, and a layer of gold disposed on a front side of the layer of palladium;
at least one main conductive trace disposed on the flexible insulating substrate in electrical contact with the plurality of conductive pads, wherein the at least one main conductive trace is arranged so that each of the conductive pads can be driven by an electrical signal; and
a plurality of flexible polymer regions, each of which is disposed as a flexible polymer layer on a front side of a respective one of the conductive pads, wherein each of the flexible polymer regions has a dielectric constant that is greater than 10 at at least one frequency between 50 kHz and 1 MHz.

20. The apparatus of claim 19, wherein the layer of nickel is electroless nickel, the layer of palladium is electroless palladium, and the layer of gold is immersion gold.
